(19) United States
(12) Patent Application Publication
(10) Pub. No.: US 2013/0115599 A1
(43) Pub. Date: May 9, 2013

(54) INCREASED CIP2A EXPRESSION AND BLADDER CANCER IN HUMANS

(71) Applicant: Medical Diagnostic Laboratories, LLC, Hamilton, NJ (US)

(72) Inventors: Lisa P. Huang, Princeton, NJ (US); Jason Trama, Burlington, NJ (US)

(73) Assignee: MEDICAL DIAGNOSTIC LABORATORIES, LLC, Hamilton, NJ (US)

(21) Appl. No.: 13/670,787

(22) Filed: Nov. 7, 2012

Related U.S. Application Data
(60) Provisional application No. 61/628,917, filed on Nov. 8, 2011.
(60) Provisional application No. 61/660,998, filed on Jun. 18, 2012.

(51) Int. Cl. G01N 33/574; C12Q 1/68; G01N 21/64; G01N 21/76

(52) U.S. Cl. 435/6.11; 435/7.9; 435/7.92; 435/6.12

(57) ABSTRACT

The present invention provides a method of detecting CIP2A protein in a bladder tissue. Methods and compositions are provided herein for detecting and diagnosing bladder cancer by obtaining a bladder tissue from a human subject suspected of bladder cancer, followed by detecting CIP2A protein or mRNA levels in the bladder tissue using Western blot analysis or ELISA to specifically detect CIP2A protein or qRT-PCR to specifically detect CIP2A mRNA. The present method permits specific detection of CIP2A protein or mRNA in bladder tissue as a biomarker for bladder cancer in humans.

### CROSS-REFERENCE TO RELATED APPLICATIONS

The present application claims the benefit of priority under 35 U.S.C. §119(e) to U.S. Provisional Application No. 61/628,918 filed Nov. 9, 2011 and U.S. Provisional Application No. 61/660,998 filed Jun. 18, 2012, the disclosures of which are hereby incorporated by reference in their entireties.

### FIELD OF INVENTION

The present invention generally relates to the increased expression of an oncoprotein in bladder cancer in humans. Specifically, the present invention provides a method for detecting the increased protein expression or mRNA expression of CIP2A in bladder tissues and methods of using same in predicting the presence of bladder cancer.

BACKGROUND OF THE INVENTION

Bladder cancer is the fourth most common cancer diagnosed in men. In 2011, National Cancer Institute estimates approximately 70,000 cases of bladder cancer will be diagnosed; of those, more than 15,000 are expected to die. According to the American Cancer Society, the five-year survival rate for patients diagnosed with bladder cancer is 98% (at stage 0), 88% (at stage I), 63% (at stage II), 46% (at stage III), and 15% (at stage IV). These bleak statistics highlight the fact that early detection of bladder cancer is critical for the intervention of the disease.

Early detection of bladder cancer allows preservation of the bladder, attenuation of complications such as bleeding or infections, prevention of metastasis and hence long-term survival. In most cases, hematuria (blood in urine) is the first warning sign of bladder cancer. The standard methods to detect bladder cancer include: 1) urinalysis; 2) urine cytology; and 3) urine test for tumor biomarkers (such as UroVysion™, BTA tests, Immunocyt™, NMP22 BladderChek®).

Numerous urine-based markers have been tested for bladder cancer detection and surveillance. A bladder tumor-associated antigen assay (BTA-Stat/TRAK) detects the presence of human complement factor H related protein in urine as a biomarker for bladder cancer. This test has a reported sensitivity of 67% and specificity of 70%. Another immunoassay is available to detect biomarkers such as mucin and carcinoembryonic antigen found on cancer cells. Elevated levels of a nuclear matrix protein (NMP22) in urine offer diagnosis of bladder cancer. Genetic detection of aneuploidy for chromosomes 3, 7 and 17, and loss of the 9p21 locus using fluorescence in situ hybridization technology (e.g., UroVysion™) provides an initial diagnosis of bladder carcinoma in patients with hematuria. This test has a reported sensitivity of 71.0% and specificity of 65.8%.

There is a continuing need to search and identify novel biomarkers that can detect bladder cancer at its early stage. The present invention provides a novel assay to detect an oncoprotein (i.e., CIP2A) in bladder tissues and offers a good tool for bladder cancer detection in humans.

### SUMMARY OF THE INVENTION

The present invention provides a method of detecting the CIP2A expression in a bladder tissue obtained from a human which comprises the steps of obtaining a bladder tissue from a human suspected of bladder cancer, and quantifying either the CIP2A protein expression, or mRNA expression in said tissue. An elevated CIP2A expression level in the bladder tissue is indicative of bladder cancer in humans.

In one embodiment, the CIP2A expression is detected by quantifying the CIP2A protein level in a bladder tissue. In another embodiment, the present invention provides a method of detecting CIP2A protein expression in a bladder tissue obtained from a human suspected of bladder cancer, comprising the steps of: (a) obtaining a bladder tissue from a human; (b) preparing a lysate from said bladder tissue; and (c) quantifying CIP2A protein expression in said prepared lysate, wherein an increased CIP2A protein expression in said prepared lysate relative to that in a normal bladder tissue is indicative of bladder cancer in said human. Preferably, a lysate is prepared from the bladder tissue. The lysate is prepared using a modified RIPA solution. Preferably, the volume/volume ratio of tissue volume and RIPA volume is 1:1.

In another embodiment, the detecting step of the CIP2A protein expression is performed using a Western blot assay or an ELISA.

In another embodiment, the CIP2A expression is detected by quantifying mRNA level in a bladder tissue. Preferably, mRNA is isolated from a bladder tissue, followed by mRNA quantification by qRT-PCR.

In another embodiment, the present invention provides a method of detecting CIP2A mRNA expression in a bladder tissue obtained from a human suspected of bladder cancer, comprising the steps of: (a) obtaining a bladder tissue from a human;

(b) isolating mRNA from said bladder tissue; and (c) quantifying CIP2A mRNA expression in said bladder tissue, wherein an increased CIP2A expression in said bladder tissue relative to that from a normal bladder tissue is indicative of bladder cancer in said human. Preferably, mRNA is isolated from a bladder tissue with guanidinium thiocyanate or phenol-chloroform.

In another embodiment, the detecting step of the CIP2A mRNA expression is performed using a qRT-PCR.

In yet another embodiment, the present invention provides a kit for quantifying CIP2A expression in a bladder tissue from a human suspected of bladder cancer, comprising: (a) reagents for quantifying CIP2A expression level; and (b) an instruction for using same reagents. In another embodiment, the reagents comprise of an anti-CIP2A that is used in quantification of CIP2A protein expression. In another embodiment, the reagents comprise of a primer pair and a hybridization probe that are used in quantification of CIP2A mRNA expression.

### BRIEF DESCRIPTION OF THE DRAWINGS

FIG. 1 depicts Western blot analysis showing the detection of CIP2A protein expression in HeLa cells using three (3) anti-CIP2A antibodies (i.e., SC-80662 (mAb), SC-80660 (mAb), and A301-454A (pAb)). Specificity of the antibody binding is revealed in cells that were stably transfected with shRNA (i.e., CIP2A protein in these cells was knocked down) (See, Experimental Materials & Procedures). (−) lane represents control HeLa cells; (+) lane represents HeLa cells that were stably transfected with CIP2A shRNA. mAb: monoclonal antibody; pAb: polyclonal antibody.

FIG. 2 depicts Western blot analysis showing the expression of CIP2A protein in bladder cells. CIP2A was found to be abundantly expressed in four (4) bladder cancer cell lines (i.e., RT-4, T-24, 5637, and TCCSUP). Note that CIP2A was also found expressed in bladder epithelial cells (i.e., UroTSA). No CIP2A expression was found in colon cells (i.e., CCD112-CoN) as well as ectocervical cells (i.e., Ect1). β-actin was used as a loading control.

FIG. 3 depicts the expression of CIP2A protein in bladder tissue lysates derived from patients with bladder cancer. In this study, five (5) bladder tumor tissues (i.e., T) from patients with bladder cancer were examined as well as their adjacent normal tissues (i.e., N) from the same patients and were used as a comparison. Note that four (4) out of the five (5) bladder tissues from bladder cancer patients expressed CIP2A. HeLa cells serves as control. β-actin serves as a loading control. N: Normal; T: Tumor.

FIG. 4 depicts the expression of CIP2A in bladder tissue lysates derived from patients with bladder cancer. In this study, eight (8) bladder tumor tissues (i.e., T) from patients with bladder cancer were examined as well as their adjacent normal tissues (i.e., N) from the same patients and were used as a comparison. Note that seven (7) out of eight (8) bladder tissues from bladder cancer patients expressed CIP2a. HeLa cells serves as control. β-actin serves as a loading control. N: Normal; T: Tumor.

FIG. 5 depicts the expression of CIP2A in bladder tissue lysates derived from patients with bladder cancer. In this study, seven (7) bladder tissues (i.e., T) from patients with bladder cancer were studies as well as their adjacent normal tissues (i.e., N) from the same patients and were used as a comparison. Note that four (4) out if seven (7) bladder tissues from bladder patients expressed CIP2A. HeLa serves as a control cell line. β-actin serves as a loading control protein. N: Normal; T: Tumor.

FIG. 6 depicts the detection of CIP2A in urine after spiking with recombinant human CIP2A. Note that 0.8 ng to 500 ng of recombinant human CIP2A (i.e., FLAG-CIP2A) were spiked in neat human urine followed by Western blot analysis using sc-80662 mAb.

FIG. 7 depicts the detection of CIP2A in spiked urine after ˜500 fold concentration (i.e., acetone and filter concentrations) followed by Western blot analysis using sc-80662. Note that the optimized condition detects as little as 50 ng of CIP2A in spiked urine.

FIG. 8 depicts the detection of CIP2A in urine obtained from patients suffering from TCC bladder cancer. UroTSA cell line was used as a positive control. Note that none of the ten (10) urine samples from bladder cancer patients showed a detectable CIP2A in a Western blot. In contrast, bladder tissues from some of these patients (e.g., MDL-719, MDL-038, and MDL-847) showed an increase in CIP2A expression.

### DETAILED DESCRIPTION OF THE INVENTION

The present invention can be better understood from the following description of preferred embodiments, taken in conjunction with the accompanying drawings. It should be apparent to those skilled in the art that the described embodiments of the present invention provided herein are merely exemplary and illustrative and not limiting.

Definitions:

The following terms shall have the meanings as defined hereunder:

As used herein, the term “CIP2A” (known as Cancerous Inhibitor of PP2A) refers to the protein that inhibits PP2A tumor suppressor activity. The CIP2A protein has the amino acid sequence set forth in NCBI Accession No: NP_{—}065941, the disclosure of which is herein incorporated by reference. The CIP2A protein is encoded by the KIAA1524 gene, whose nucleotide sequence is set forth in NCBI Accession No: BC136371, the disclosure of which is herein incorporated by reference.

As used herein, the term “PP2A” refers to protein-phosphatase 2 (also known as PP2). PP2A is an enzyme in humans that is encoded by the PPP2CA gene. Structurally, PP2A consists of three (3) subunits: (i) structural A and (ii) catalytic C subunits, and (iii) a regulatory B subunit.

As used herein, the term “shRNA” refers to small hairpin RNA or short hairpin RNA. shRNA is a sequence of RNA that makes a tight hairpin turn that can be used to silence gene expression via RNA interference. For purposes of this application, CIP2A shRNA is used to silence CIP2A gene expression. The CIP2A shRNA used is obtained from a commercial source (Thermo Scientific Open Biosystems).

As used herein, the term “lysis” refers to the breaking down of a cell or tissue, often by enzymatic or osmotic mechanisms that compromise its integrity. A fluid containing the contents of lysed cells or tissues is called “lysate.”

As used herein, the term “Western blot assay” refers to an analytical technique used to detect specific proteins in a given sample of tissue homogenate or extract. It utilizes gel electrophoresis to separate either native proteins or denatured proteins by their lengths or 3-D structures. The separated proteins are transferred to a membrane (typically nitrocellulose or PVDF), and are detected using antibodies specific against a target protein.

As used herein, the term “tissue” refers to a cellular organizational level intermediate between cells and a complete organism. A tissue is an ensemble of cells, not necessarily identical, but from the same origin, that together carry out a specific function. A bladder tissue refers to a tissue obtained from a bladder.

As used herein, the term “antibody” refers to an immunoglobulin produced by B cells and has structural units of two large heavy chains and two small light chains. There are two general classes of antibody; namely, monoclonal antibody and polyclonal antibody. Monoclonal antibodies (mAb) refer to monospecific antibodies that are the same because they are made by identical immune cells that are all clones of a unique parent cell. Monoclonal antibodies are typically made by fusing myeloma cells with the spleen cells from a mouse that has been immunized with the desired antigen. Polyclonal antibodies are antibodies obtained from different B cells. They are a combination of immunoglobulins secreted against a specific antigen, each identifying a different epitope. Animals frequently used for polyclonal antibody production include goats, guinea pigs, rabbits, horses, sheep and the like. Rabbit is the most commonly used laboratory animal for this purpose.

As used herein, the term “protein” refers to a chain of at least two amino acids. The terms “polypeptide,” “peptide,” or “protein” are used interchangeably.

As used herein, the term “sample” refers to a body sample in which biomarkers can be detected. For purposes of this application, a sample refers to biopsy tissues of bladder collected from an individual suspected of bladder cancer.

As used herein, the term “expression level” refers to expression of protein as measured quantitatively by methods such as Western blot or ELISA. The term “expression” also encompasses expression of CIP2A mRNA level as measured quantitatively by methods including but not limited to, for example, qRT-PCR.

As used herein, the term “detect an expression level” refers to measuring or quantifying either protein expression or mRNA expression.

As used herein, the term “an increased expression level” refers to increased protein expression level or mRNA expression level relative to a normal value. CIP2A protein is notably absent in normal bladder tissues or cultured cells. For purposes of this application, an increased CIP2A protein expression refers to an elevated CIP2A protein in bladder tissue above that present in a normal bladder tissue from a control subject (free of bladder cancer) or that present in adjacent normal bladder tissue from a bladder cancer patient. Normal bladder tissues expressed a basal low CIP2A mRNA level. For purposes of this application, an increased CIP2A mRNA expression refers to an elevated CIP2A mRNA in a bladder tissue above that present in a normal bladder tissue from a control subject (free of bladder cancer) or adjacent normal bladder tissue from a bladder cancer patient.

As used herein, the term “bladder cancer” refers to a cancerous tumor in the bladder. For purposes of this application, bladder cancer is not intended to be limited to cancer of any specific types (i.e., include many types of cancer in the bladder such as transitional cell carcinoma (TCC), squamous cell carcinoma, adenocarcinoma and combinations thereof).

As used herein, the term “TCC” refers to transitional cell carcinoma (also known as urothelial cell carcinoma or UCC). It is a type of cancer that typically occurs in the urinary system: the kidney, urinary bladder, and accessory organs. It is the most common type of bladder cancer and cancer of the ureter, urethra, and urachus. TCC often arises from the transitional epithelium, a tissue lining the inner surface of these hollow organs.

As used herein, the term “stage” in reference to bladder cancer refers to the degree of tumor invasion into the bladder wall. There are carcinoma in-situ (Tis) and Stages Ta, T1, T2, T3 and T4. Ta and T1 represent superficial bladder cancers and they are restricted to the inner epithelial lining of the bladder and do not involve muscle walls. Stage Ta is confined to mucosa. Stage T1 superficially invades lamina propria and is regarded as more aggressive than Ta. Stages T2, T3 and T4 represent invasive tumors that have extended into muscle (Stage T2) and perivesical fat layer beyond the muscle (Stage T3) as well as Stage T4 (metastatic tumors) that have invaded into local nodes or distant organs. For purposes of this application, “high-stage” encompasses T3 and T4.

As used herein, the term “grade” in reference to bladder cancer relates to the degree of cellular differentiation and histological morphology. Grade 1 refers to well differentiated, having an existing papillary architecture, fine chromatin, and a little indication of nucleoli or mitoses. Grade 2 refers to moderately differentiated, having a papillary architecture, granular chromatin, and a stronger indication of nucleoli and mitoses. Grade 3 refers to poorly differentiated, least likely having a papillary architecture, have coarse chromatin, and have many examples of nucleoli and mitoses. For purposes of this application, “high-grade” encompasses Grade 3.

As used herein, the term “UroTSA” refers to a cell line isolated from a primary culture of normal human urothelium through immortalization with a construct containing the SV40 large T antigen. It proliferates in serum-containing growth medium as a cell monolayer with little evidence of uroepithelial differentiation.

As used herein, the term “RT-4” refers to a human urinary bladder papilloma cell line which was isolated from a 63-year old Caucasian male. This cell line grows as a cell monolayer in a growth medium supplemented with fetal bovine serum.

As used herein, the term “T-24” refers to a human bladder cancer cell line that has been established from a highly malignant grade III human urinary bladder carcinoma of an 81-year old Caucasian male patient. This cell line grows as a cell monolayer in a growth medium supplemented with fetal bovine serum.

As used herein, the term “5637” refers to a bladder cancer cell line that has been established from the primary grade II bladder carcinoma of a 68-year old Caucasian male patient. This cell line grows as a cell monolayer in a growth medium supplemented with fetal bovine serum.

As used herein, the term “UCCSUP” refers to a bladder cancer cell line that was isolated in from an anaplastic transitional cell carcinoma in the neck of the urinary bladder. This cell line grows as a cell monolayer in a growth medium supplemented with fetal bovine serum.

As used herein, the term “CCD112-CoN” refers to normal colon cell line (also known as CCD-112 CoN). This cell line grows as a cell monolayer in a growth medium supplemented with fetal bovine serum.

As used herein, the term “Ect1” refers to a cell line isolated from normal epithelial tissue of a premenopausal woman undergoing hysterectomy for endometriosis through immortalization with a construct containing E6/E7 oncogenes.

The present invention provides a novel biomarker for detection of bladder cancer diseases. In particular, the present inventors discovered the use of CIP2A as a biomarker to detect and diagnose bladder cancer in humans. Using clinical samples, the present inventors demonstrated that CIP2A increases its expression levels (either protein level or mRNA level) in bladder tissues derived from patients who suffer from bladder cancer as compared to bladder tissues derived from control patients who do not have bladder cancer. The increased expression levels of CIP2A (either protein or mRNA) maintains throughout various cancer stages (i.e., stage I, stage II, and stage III of bladder cancer).

The present invention provides a diagnostic assay with a high sensitivity and specificity in detecting and predicting bladder cancer occurrence in a human suspected of having bladder cancer.

There has been a long-felt need for identifying novel biomarkers that would permit early detection and diagnosis of bladder diseases. According to American Cancer Society in 2005, more than 63,000 bladder tumors would be diagnosed and more than 13,000 people would die of the disease. Men are more at risk than women. The risk is greater for people older than 60 and those who have been exposed to environmental or occupational toxins. Early detection greatly improves the chances of survival. If identified early, 95% of bladder cancer patients survive at least five years—a time period commonly used when discussing cancer survival. However, if bladder cancer is found after it spreads beyond the superficial layers of the lining of the bladder, five-year survival rates drop significantly.

Diagnosing bladder cancer, especially after seeing blood in the urine (the most common sign), is done by various methods. These include directly visualizing the bladder through cystoscopy. The procedure utilizes an attached tiny camera to visualize the inside of the bladder. A number of tests can be performed on urine samples to aid the diagnosis of bladder cancer. These tests include the bladder-tumor-associated antigen test, the BTA stat test, the BTA TRAK® test, the fibrin/fibrinogen degradation products (FDP®) test, and the NMP22™ assay. The BTA® test was designed to detect proteins that are released by reproduction of bladder tumor cells, and its interpretation does not require a technician or specialist. The BTA® test significantly identifies superficial (surface) bladder tumors by changing color. The top of the BTA® test strip turns yellow when positive for bladder cancer, and it turns green when negative. The BTA stat test is an immunologic assay that can be used to identify recurrent bladder cancer. The FDP® test detects the breakdown products of blood-clotting proteins (fibrin, fibrinogen), which are increased in the urine in the presence of bladder cancer.

Another test involves the NMP22™ assay which measures specific proteins from the nuclear matrix (cell center). It is reported to detect transitional cell carcinoma (TCC) with a sensitivity of roughly 67%. NMP22™ assay has been approved by FDA to help initial diagnosis of bladder cancer. Other bladder cancer tests include, for example, microsatellite DNA analysis between urine cells and unaffected cells (such as lymphocytes) from the same patient. The accuracy of the test, however, has not been proven. To date, there exists a limited number of molecular biomarkers commercially available.

The present inventors cured the prior art deficiency and fulfilled the long-felt need in this medical area. The present inventors surprisingly discovered CIP2A as a biomarker for detection of bladder diseases in human. CIP2A is a recently discovered protein that is shown to inhibit PP2A tumor suppressor activity in human malignancies (Junttila M R et al., Cell 130(1): 51-62, 2007). To the best of the present inventors' knowledge, CIP2A has been associated with three (3) types of human malignancies;

namely, (i) head and neck squamous cell carcinoma, (ii) colon cancer, and (iii) gastric cancer. The exact mechanism of action for CIP2A in neoplasia is unclear. CIP2A is known to inhibit PP2A activity toward c-myc serine 62 (S62), and affect its proteolygic degradation.

The present invention represents the first report linking CIP2A expression with bladder cancer. The present invention provides a novel and non-obvious finding that CIP2A is a good molecular biomarker for detection of bladder cancer. The present finding is unexpected because the present assay employing CIP2A expression provides a high sensitivity and specificity. Contrary to our expectation, CIP2A protein does not change in urine (i.e., release into urine) from patients suffering from bladder diseases. The finding that CIP2A expression is increased in bladder tissue and not urine provides specificity.

Collection and Preparation Biological Samples

Biological samples of the bladder in humans (i.e., bladder tissues) can be conveniently collected by methods known in the art. Usually, a bladder tissue can be harvested by trained medical staffs or physicians under sterile environment. Bladder tissue biopsies often are taken, for example, by endoscopic means. After harvested from patients, biological samples may be immediately frozen (under liquid nitrogen) or put into a storage, or transportation solution to preserve sample integrity. Such solutions are known in the art and commercially available, for example, UTM-RT transport medium (Copan Diagnostic, Inc, Corona, Calif.), Multitrans Culture Collection and Transport System (Starplex Scientific, Ontario, CN), ThinPrep® Paptest Preservcyt® Solution (Cytyc Corp., Boxborough, Mass.) and the like.

A. Sample Preparation: Protein Extraction

After collection, biological samples are prepared prior to detection of biomarkers. Sample preparation includes isolation of protein or nucleic acids (e.g., mRNA). These isolation procedures involve separation of cellular protein or nucleic acids from insoluble components (e.g., cytoskeleton) and cellular membranes.

In one embodiment, bladder tissues are treated with a lysis buffer solution prior to isolation of protein or nucleic acids. A lysis buffer solution is designed to lyze tissues, cells, lipids and other biomolecules potentially present in the raw tissue samples. Generally, a lysis buffer of the present invention may contain one or more of the following ingredients: (i) chaotropic agents (e.g., urea, guanidine thiocyanide, or formamide); (ii) anionic detergents (e.g., SDS, N-lauryl sarcosine, sodium deoxycholate, olefine sulphates and sulphonates, alkyl isethionates, or sucrose esters); (iii) cationic detergents (e.g., cetyl trimethylammonium chloride); (iv) non-ionic detergents (e.g., Tween®-20, polyethylene glycol sorbitan monolaurate, nonidet P-40, Triton® X-100, NP-40, N-octyl-glucoside); (v) amphoteric detergents (e.g., CHAPS, 3-dodecyl-dimethylammonio-propane-1-sulfonate, lauryldimethylamine oxide); or (vi) alkali hydroxides (e.g., sodium hydroxide or potassium hydroxide). Suitable liquids that can solubilize the cellular components of biological samples are regarded as a lysis buffer for purposes of this application.

In another embodiment, a lysis buffer may contain additional substances to enhance the properties of the solvent in a lysis buffer (e.g., prevent degradation of protein or nucleic acid components within the raw biological samples). Such components may include proteinase inhibitors, RNase inhibitors, DNase inhibitors, and the like. Proteinase inhibitors include but not limited to inhibitors against serine proteinases, cysteine proteinases, aspartic proteinases, metallic proteinases, acidic proteinases, alkaline proteinases or neutral proteinases. RNase inhibitors include common commercially available inhibitors such as SUPERase.In™ (Ambion, Inc. Austin, Tex.), RNase Zap® (Ambion, Inc. Austin, Tex.), Qiagen RNase inhibitor (Valencia, Calif.), and the like.

B. Sample Preparation: Nucleic Acid Extraction

Nucleic acids, such as mRNA, can be conveniently extracted from biological samples obtained from bladder tissues using standard extraction methods that are known in the art. Standard extraction methods include guanidinium thiocyanate, phenol-chloroform extraction, guanidine-based extraction, and the like. Commercial nucleic acid extraction kits may be employed. For example, RNeasy Fibrous Tissue Mini Kit from Qiagen (Valencia, Calif.) and RNAimage Kit from GenHunter Corporation (USA).

Detection of CIP2A Protein Expression Level

After protein extraction, expression level of CIP2A biomarker in the biological samples can be determined using standard assays that are known in the art. These assays include, but not limited to Western blot analysis, ELISA, radioimmunoassay, immune-histochemistry assay, and the like. In a preferred embodiment, expression level of CIP2A biomarkers may be detected by Western blot analysis.

Western Blot

After cellular proteins are extracted or isolated from the biological samples (e.g., bladder tissues), the cellular proteins are separated using SDS-PAGE gel electrophoresis. The conditions for SDS-PAGE gel electrophoresis can be conveniently optimized by one skilled in the art.

Protein biomarkers in the gels can then be transferred onto a surface such as nitrocellulose paper, nylon membrane, PVDF membrane and the like. The conditions for protein transfer after SDS-PAGE gel electrophoresis may be optimized by one skilled in the art. Preferably, a PVDF membrane is used.

To detect the biomarker proteins, a first antibody specific for CIP2A is employed. Bound cellular proteins (e.g., 50-100 μg) on the membrane are incubated with a first antibody in a solution. An optimized first antibody concentration (e.g., 0.2-2 μg/mL) may be used. Incubation conditions may be optimized to maximize binding of the first antibody with the bound biomarker proteins. For example, 1 μg/mL of the first antibody is used and incubation time is 1-6 hours. Preferably, the incubation time is 2 hours. The first antibody may either be a monoclonal antibody or polyclonal antibody. Antibodies against the various protein biomarkers can be prepared using standard protocols or obtained from commercial sources. Techniques for preparing mouse monoclonal antibodies or goat or rabbit polyclonal antibodies (or fragments thereof) are well known in the art. Optionally, the membrane is incubated with a blocking solution before the incubation with the first antibody. The blocking solution may include agents that reduce non-specific binding of antibody. An exemplary blocking solution may include 5% skim milk in PBST (0.1% Tween-20).

After the incubation with the first antibody, the unbound antibody is removed by washing. An exemplary washing solution includes PBST. Protein biomarker-first antibody complex can be detected by incubation with a second antibody that is specific for the first antibody. The second antibody may be a monoclonal antibody or a polyclonal antibody (e.g., mouse, rabbit, or goat). The second antibody may carry a label that may be a directly detectable label or may be a component of a signal-generating system. Preferably, the second antibody is a goat anti-rabbit antibody or goat anti-mouse antibody that is labeled with a peroxidase. Such labeled antibodies and systems are well known in the art.

Direct detectable label or signal-generating systems are well known in the field of immunoassay. Labeling of a second antibody with a detectable label or a component of a signal-generating system may be carried out by techniques well known in the art. Examples of direct labels include radioactive labels, enzymes, fluorescent and chemiluminescent substances. Radioactive labels include ¹²⁴I, ¹²⁵I, ¹²⁸I, ¹³¹I, and the like. A fluorescent label includes fluorescein, rhodamine, rhodamine derivatives, and the like. Chemiluminescent substances include ECL chemiluminescent.

ELISA

In another embodiment, detection and quantification of CIP2A protein level is determined by ELISA.

In a typical ELISA, a first antibody is immobilized onto a solid surface. Immobilization of the first antibody may be performed on any inert support useful in immunological assays. Examples of inert support include sephadex beads, polyethylene plates, polypropylene plates, polystyrene plates, and the like. In one embodiment, the first antibody is immobilized by coating the antibody on a microtiter plate. In another embodiment, the microtiter plate is a microtest 96-well ELISA plate, such as those sold under the name Nunc Maxisorb or Immulon.

The first antibody is an antibody specific (to bind or to recognize) the protein biomarkers of interest. The first antibody may either be a monoclonal antibody, polyclonal antibody, or a fragment thereof. The first antibody may be acquired via commercial sources, or prepared by standard protocols well known in the art. A solid surface includes a 96-well plate.

CIP2A biomarker present in a biological sample is captured by immobilizing a first antibody onto a surface. To do so, a protein extract from biological samples is incubated with the immobilized first antibody. Conditions for incubation can be optimized to maximize the formation of protein biomarker-first antibody complex. Preferably, an incubation time of 2-8 hours and a temperature of 25° C. may be used. Unbound first antibody is removed by washing.

To detect the formation of protein biomarker-first antibody complex, a second antibody is used. The second antibody may either be a monoclonal antibody or polyclonal antibody. Preferably, the second antibody is a polyclonal antibody, derived from goat or rabbit. Preparation of the second antibody is in accordance with established protocol or commercially available. Incubation of the second antibody can conveniently be optimized to maximize the binding. Preferably, an incubation time of 2-8 hours and a temperature of 25° C. may be used. Unbound second antibody is easily removed by washing. The second antibody is either directly labeled or conjugated with a signal-generating system.

The methods of detecting the presence of a directly labeled second antibody or a second antibody conjugated with a signal-generating system are well known to those of skill in the art. Suitable direct labels include moieties such as fluorophores, radioactive labels, and the like. Examples of radioactive labels include but not limited to ³²P, 14C, ¹²⁵I, ³H, and ¹³¹I. Examples of fluorophores include but not limited to fluorescein, rhodamine, and the like.

The second antibody may conveniently be conjugated to a signal-generating system such as an enzyme. Exemplary enzymes include horseradish peroxidase (HRP), alkaline phosphatase, and the like. The conjugation of an enzyme to the second antibody is a standard manipulative procedure for one of ordinary skill in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166. Detection of the presence of second antibody can be achieved simply by adding a substrate to the enzyme. The methodology of such enzyme-substrate interaction is well within one skilled in the art's capability.

Detection of CIP2A mRNA Expression Level

The present invention is directed to a discovery that CIP2A level is elevated during the pathogenesis of bladder cancer. In one embodiment, bladder CIP2A level is increased via the steady-state CIP2A mRNA expression levels. Detection of CIP2A mRNA expression levels includes standard mRNA quantitation assays that are well-known in the art. These assays include but not limited to qRT-PCR, Northern blot analysis, RNase protection assay, and the like.

In one embodiment, the present invention provides the use of qRT-PCR to detect the expression level of bladder cancer biomarkers. qRT-PCR (quantitative reverse transcription-polymerase chain reaction) is a sensitive technique for mRNA detection and quantitation. Compared to Northern blot analysis and RNase protection assay, qRT-PCR can be used to quantify mRNA levels from much smaller samples.

Real-time polymerase chain reaction, also called quantitative real time polymerase chain reaction (Q-PCR/qPCR/qRT-PCR), is used to amplify and simultaneously quantify a targeted DNA molecule. It enables both detection and quantification (as absolute number of copies or relative amount when normalized to DNA input or additional normalizing genes) of one or more specific sequences in a DNA sample. Currently at least four (4) different chemistries, TaqMan® (Applied Biosystems, Foster City, Calif.), Molecular Beacons, Scorpions® and SYBR® Green (Molecular Probes), are available for real-time PCR.

All of these chemistries allow detection of PCR products via the generation of a fluorescent signal. TaqMan probes, Molecular Beacons and Scorpions depend on Förster Resonance Energy Transfer (FRET) to generate the fluorescence signal via the coupling of a fluorogenic dye molecule and a quencher moiety to the same or different oligonucleotide substrates. SYBR Green is a fluorogenic dye that exhibits little fluorescence when in solution, but emits a strong fluorescent signal upon binding to double-stranded DNA.

Two common methods for detection of products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target.

Real-time PCR, when combined with reverse transcription, can be used to quantify messenger RNA (mRNA) in cells or tissues. An initial step in the reverse transcription PCR amplification is the synthesis of a DNA copy (i.e., cDNA) of the region to be amplified. Reverse transcription can be carried out as a separate step, or in a homogeneous reverse transcription-polymerase chain reaction (RT-PCR), a modification of the polymerase chain reaction for amplifying RNA. Reverse transcriptases suitable for synthesizing a cDNA from the RNA template are well known.

Following the cDNA synthesis, methods suitable for PCR amplification of ribonucleic acids are known in the art (See, Romero and Rotbart in Diagnostic Molecular Biology: Principles and Applications pp. 401-406). PCR reagents and protocols are also available from commercial vendors, such as Roche Molecular Systems. PCR can be performed using an automated process with a PCR machine.

Primer sets used in the present qRT-PCR reactions for various biomarkers may be prepared or obtained through commercial sources. For purposes of this application, the primer sets used in this invention include primers ordered from Abi (Assay ID, HS00405413_ml) (Foster City, Calif.). The primers used in the PCR amplification preferably contain at least 15 nucleotides to 50 nucleotides in length. More preferably, the primers may contain 20 nucleotides to 30 nucleotides in length. One skilled in the art recognizes the optimization of the temperatures of the reaction mixture, number of cycles and number of extensions in the reaction. The amplified product (i.e., amplicons) can be identified by gel electrophoresis.

Aided with the help of DNA probe, the real-time PCR provides a quantum leap as a result of real-time detection. In real-time PCR assay, a fluorometer and a thermal cycler for the detection of fluorescence during the cycling process is used. A computer that communicates with the real-time machine collects fluorescence data. This data is displayed in a graphical format through software developed for real-time analysis.

In addition to the forward primer and reverse primer (obtained via commercial sources), a single-stranded hybridization probe is also used. The hybridization probe may be a short oligonucleotide, usually 20-35 by in length, and is labeled with a fluorescent reporting dye attached to its 5′-end as well as a quencher molecule attached to its 3′-end. When a first fluorescent moiety is excited with light of a suitable wavelength, the absorbed energy is transferred to a second fluorescent moiety (i.e., quencher molecule) according to the principles of FRET. Because the probe is only 20-35 by long, the reporter dye and quencher are in close proximity to each other and little fluorescence is detected. During the annealing step of the PCR reaction, the labeled hybridization probe binds to the target DNA (i.e., the amplification product). At the same time, Taq DNA polymerase extends from each primer. Because of its 5′ to 3′ exonuclease activity, the DNA polymerase cleaves the downstream hybridization probe during the subsequent elongation phase. As a result, the excited fluorescent moiety and the quencher moiety become spatially separated from one another. As a consequence, upon excitation of the first fluorescent moiety in the absence of the quencher, the fluorescence emission from the first fluorescent moiety can be detected. By way of example, a Rotor-Gene System is used and is suitable for performing the methods described herein. Further information on PCR amplification and detection using a Rotor-Gene can conveniently be found on Corbett's website.

In another embodiment, suitable hybridization probes such as intercalating dye (e.g., Sybr-Green I) or molecular beacon probes can be used. Intercalating dyes can bind to the minor grove of DNA and yield fluorescence upon binding to double-strand DNA. Molecular beacon probes are based on a hairpin structure design with a reporter fluorescent dye on one end and a quencher molecule on the other. The hairpin structure causes the molecular beacon probe to fold when not hybridized. This brings the reporter and quencher molecules in close proximity with no fluorescence emitted. When the molecular beacon probe hybridizes to the template DNA, the hairpin structure is broken and the reporter dye is no long quenched and the real-time instrument detects fluorescence.

The range of the primer concentration can optimally be determined. The optimization involves performing a dilution series of the primer with a fixed amount of DNA template. The primer concentration may be between about 50 nM to 300 nM. An optimal primer concentration for a given reaction with a DNA template should result in a low Ct-(threshold concentration) value with a high increase in fluorescence (5 to 50 times) while the reaction without DNA template should give a high Ct-value.

The probes and primers of the invention can be synthesized and labeled using well-known techniques. Oligonucleotides for use as probes and primers may be chemically synthesized according to the solid phase phosphoramidite triester method first described by Beaucage, S. L. and Caruthers, M. H., 1981, Tetrahedron Letts., 22 (20): 1859-1862 using an automated synthesizer, as described in Needham-VanDevanter, D. R., et al. 1984, Nucleic Acids Res., 12: 6159-6168. Purification of oligonucleotides can be performed, e.g., by either native acrylamide gel electrophoresis or by anion-exchange HPLC as described in Pearson, J. D. and Regnier, F. E., 1983, J. Chrom., 255: 137-149.

Kits

The present invention provides a kit of manufacture, which may be used to perform detecting either CIP2A protein or CIP2A mRNA. The increased expression of CIP2A is shown to associate with bladder cancer. In one embodiment, an article of manufacture (i.e., kit) according to the present invention includes a set of antibodies (i.e., a first antibody and a second antibody) specific for CIP2A. Antibodies against a house-keeper gene (e.g., GADPH) are provided as a control. In another embodiment, the present kit contains a set of primers (i.e., a forward primer and a reverse primer) (directed to a region of the gene specific to the CIP2A gene and optionally a hybridization probe (directed to the same gene, albeit a different region).

Kits provided herein may also include instructions, such as a package insert having instructions thereon, for using the reagents (e.g., antibodies or primers) to quantify the protein expression level of mRNA expression level of a particular bladder cancer biomarker in a biological sample. Such instructions may be for using the primer pairs and/or the hybridization probes to specifically detect mRNA of a specific gene (e.g.,

CIP2A) in a biological sample. In another, the instructions are directed to the use of antibodies (either monoclonal or polyclonal) that recognize and bind to specific bladder cancer biomarker.

In another embodiment, the kit further comprises reagents used in the preparation of the sample to be tested for protein (e.g. lysis buffer). In another embodiment, the kit comprises reagents used in the preparation of the sample to be tested for mRNA (e.g., guanidinium thiocyanate or phenol-chloroform extraction)

The following examples are provided to further illustrate various preferred embodiments and techniques of the invention. It should be understood, however, that these examples do not limit the scope of the invention described in the claims. Many variations and modifications are intended to be encompassed within the spirit and scope of the invention.

### EXPERIMENTAL STUDIES

### EXAMPLE 1

### CIP2A Protein Expression

We sought to develop a Western blot assay specifically to detect CIP2A protein expression in cells. Because HeLa cells have been reported to express CIP2A, we utilized this particular cell line in the development of our Western blot assay.

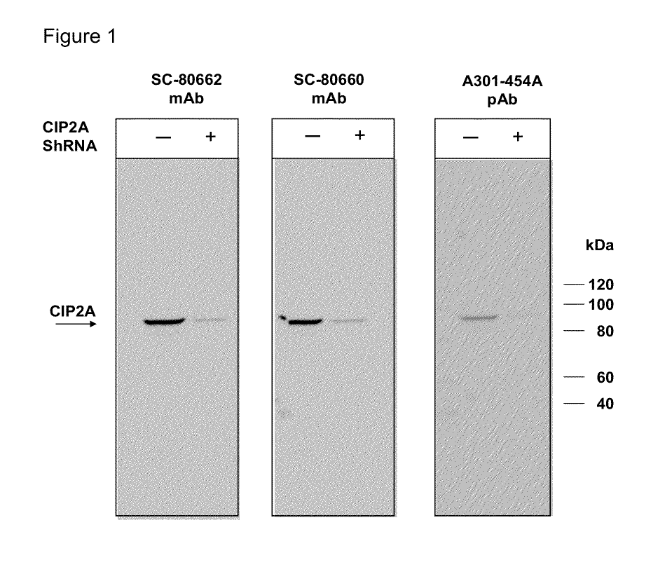

First, we cultured HeLa cells (1×10⁷ cells) and prepared cell lysate using a modified RIPA buffer. We separate cellular proteins present in cell lysate by performing SDS-PAGE. The separated cellular proteins were then transferred onto a PVDF membrane for Western blot analysis. Note that CIP2A protein (˜90 Kd) is abundantly expressed in HeLa cells. (See, FIG. 1). Our studies further revealed that both anti-CIP2A mAbs (i.e., SC-80662 and SC-80660) and pAb (i.e., A301-454A) are equally potent to detect CIP2A in our Western blot analysis.

To further examine the binding specificity of these CIP2A antibodies, we employed HeLa cells that have been stably transfected with CIP2A shRNA (i.e., CIP2A in these cells was knock down). Note that the CIP2A band was either abrogated or greatly reduced in the CIP2A shRNA HeLa cells. (See, FIG. 1), indicating binding specificity.

### EXAMPLE 2

### CIP2A is Expressed in Bladder Cells

CIP2A has been shown to express in cancer cells of liver, gastric, and breast. There is, however, no information regarding CIP2A expression in bladder cells.

We examined if CIP2A protein is expressed in bladder cancer cells using established bladder cancer cell lines. Western blot assay (described in Example 1) was used. In this series of study, we tested four (4) bladder cancer cell lines (i.e., RT-4, 5637, T-24 and TCCSUP) to evaluate if they express CIP2A. These are bladder cancer cells derived from different stages of bladder cancer of patients. Specifically, RT-4 is derived from bladder transitional cell papilloma from a patient. 5637 is derived from primary bladder carcinoma (grade II) from another patient. T-24 is derived from a highly malignant (grade III) human urinary bladder carcinoma. TCCSUP is derived from a poorly differentiated (high-grade) human bladder cancer cell line. As controls, we used: (i) normal colon cell line (i.e., CCD112-CoN), and (ii) ectocervical cell line (i.e., Ect1).

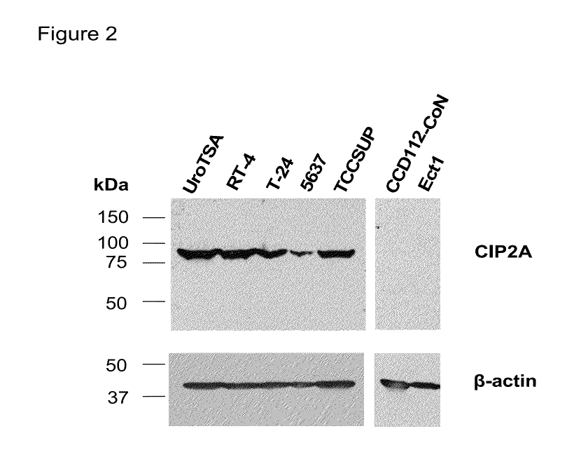

FIG. 2 shows that CIP2A protein is abundantly expressed in all four (4) bladder cancer cell lines (i.e., RT-4, 5637, T-24 and TCCSUP). This data suggests that CIP2A is expressed in cells from different bladder cancer stages. Note that CIP2A protein is not present in normal colon cells (i.e., CCD112-CoN) or ectocervical cells (i.e., Ect1). β-actin served as a loading control.

Of interest is our observation that CIP2A is also expressed in non-cancer bladder cells. For example, CIP2A is abundantly expressed in bladder epithelial cells that have been transformed with SV-40 T-antigen (UroTSA). (See, FIG. 2)

Note that we used the anti-CIP2A mAb (i.e., SC80662) in this example.

We observed the same results when the mAb SC-80660 and A301-454A were used (data not shown), confirming that CIP2A is expressed in bladder cancer cells.

All of the three (3) antibodies specifically detect CIP2A protein as a single band (See, FIG. 2) in Western blot analysis, illustrating the feasibility of the antibodies used in our study.

### EXAMPLE 3

### Increased CIP2A Expression in Bladder Cancer Tissues in Patients—Study 1

In this study, we examined if CIP2A increases its expression in bladder cancer tissues. To do so, we obtained bladder cancer tissues from patients who suffered from transitional cell carcinoma (TCC). In this series of study, we obtained five (5) bladder cancer tissues from TCC patients. Adjacent normal tissues from the same TCC subjects were obtained and used as for comparison. Western blot assay to detect CIP2A protein was performed (See, Example 1). In brief, lysate extracts were obtained from cancer tissues using modified RIPA buffer. For Western blot, 100 μg of the lysate extracts from each tissue sample were analyzed and mAb SC-80662 was used. For control, 30 μg of the HeLa cell lysate extracts were used.

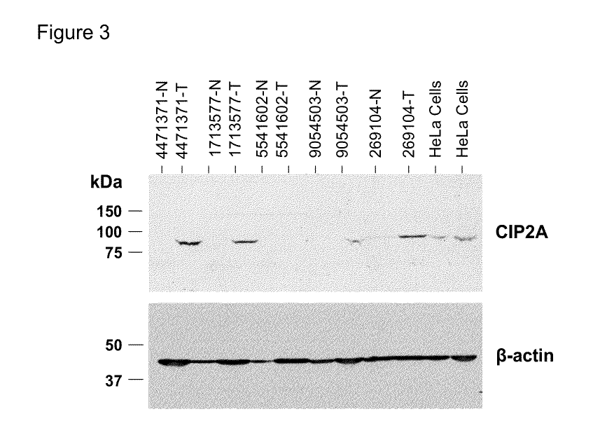

FIG. 3 shows that four (4) out of the five (5) bladder cancer tissues expressed an abundant level of CIP2A protein (i.e., 4471371-T, 1713577-T, 9054503-T, and 269104-T). Note that the match-paired adjacent normal bladder tissues did not express CIP2A protein, indicating tissue specificity for CIP2A protein expression.

### EXAMPLE 4

### Increased CIP2A Expression in Bladder Cancer Tissues in Patients—Study 2

We continued to examine CIP2A expression in bladder cancer tissues. In this series of study, we obtained eight (8) additional bladder cancer tissues in eight (TCC) patients.

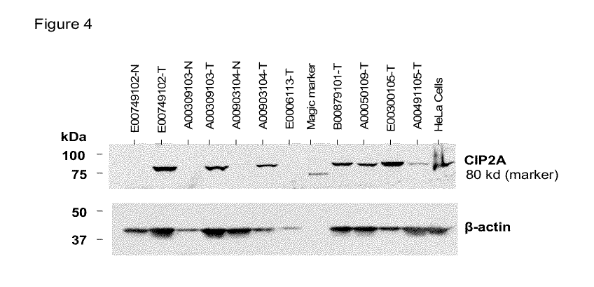

FIG. 4 shows seven (7) out of eight (8) bladder cancer tissues expressed

CIP2A protein (i.e., E0074-T, A0030-T, A0090-T, B0087-T, A0005-T, E0030T, and A0049-T). The three (3) match-paired normal adjacent bladder tissues did not express CIP2A (i.e., E0074-N, A0030-N, and A0090-N).

### EXAMPLE 5

### Increased CIP2A Expression in Bladder Cancer Tissues in Patients—Study 3

We continued to examine CIP2A expression in bladder cancer tissues. In this series of study, we obtained seven (7) additional bladder cancer tissues from seven (TCC) patients.

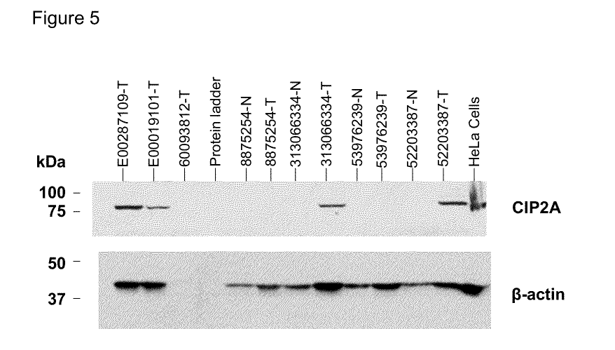

FIG. 5 shows that four (4) out of seven (7) bladder cancer tissues expressed CIP2A protein (i.e., E0028-T, E00019-T, and 31306-T). The four (4) match-paired normal adjacent bladder tissues did not express CIP2A (i.e., 8875-N, 3130-N, 5397-N, and 5220-N).

### EXAMPLE 6

### Sensitivity and Specificity of CIP2A Protein Expression in Bladder Cancer Tissues

We summarized the above-mentioned clinical studies regarding CIP2A expression in bladder cancer tissues. Table 1 provides the summary of the twenty (20) bladder cancer tissues examined with respect to: (i) the clinical diagnosis (e.g., normal vs. transitional cell carcinoma (TCC)), (ii) the pathological/histological characterization (e.g., grades and staging), and (iii) CIP2A expression.

Table 1 depicts that out of twenty (20) bladder cancer tissues CIP2A is expressed in fifteen (15) bladder cancer tissues. Of all the twelve (12) normal adjacent tissues, none express CIP2A protein.

Table 1 further depicts that CIP2A protein is expressed in both low-grade TCC and high-grade TCC. No correlation is found between CIP2A expression and bladder cancer staging in our study. In sum, the present study provides the sensitivity and specificity of CIP2A protein expression in bladder cancer tissue of 75% and 100%, respectively.

TABLE 1
CIP2A Protein Expression in Bladder Tumor Tissues

| | | | Cancer | |
|---|---|---|---|---|
| | Tissue No. | Cancer Grade | Staging | CIP2A |
| Normal | E00749102-N | | | − |
| Normal | A00309103-N | | | − |
| Normal | A00903104-N | | | − |
| Normal | 4471371-7 | | | − |
| Normal | 1713577-3 | | | − |
| Normal | 6541602-6 | | | − |
| Normal | 9054503 | | | − |
| Normal | 269104-6 | | | − |
| Normal | 8875254-N | | | − |
| Normal | 313066334-N | | | − |
| Normal | 53976239-N | | | − |
| Normal | 52203387-N | | | − |
| Cancer | 4471371-7 | High grade | T2 | + |
| Cancer | 1713577-3 | High grade | T2 | + |
| Cancer | 6541602-6 | High grade | T1 | − |
| Cancer | 269104-6 | High grade | T2 | + |
| Cancer | 52203387-T | High grade | T1 | + |
| Cancer | E00749102-T | High grade | T3 N0 M0 | + |
| Cancer | A00309103-T | High grade | T3a N2 M1 | + |
| | | | (R) | |
| Cancer | E0006113-T | High grade | T1NOS | − |
| Cancer | B00879101-T | High grade | TX NX M0 | + |
| Cancer | 8875254-T | Low grade | TA | − |
| Cancer | 313066334-T | Low grade | T1 | + |
| Cancer | 53976239-T | Low grade | T1 | − |
| Cancer | 60093812-T | Low grade | T1 | − |
| Cancer | A00903104-T | X | T2a N2 | + |
| Cancer | A00050109-T | X | T1NOS | + |
| Cancer | E00300105-T | X | T1 NOS | + |
| Cancer | A00491105-T | X | T2b No M0 | + |
| Cancer | E00287109-T | X | T4 | + |
| Cancer | E00019101-T | X | Metastatic | + |
| | | | TCC | |
| Cancer | 9054503 | | | + |
| | | | Normal tissue | 0% POS |
| | | | (N = 12) | (0/12) |
| | | | Cancer tissue | 75% POS |
| | | | (N = 20) | (15/20) |

EXAMPLE 7

### Confirmation of CIP2A Protein Expression Using Different Antibody

In the clinical study described in Example 6, we have used mAb SC-80662 to examine CIP2A expression in bladder tissues from subjects suffering from bladder cancer. In this Example, we repeated the study using two (2) different antibodies (i.e., SC-80660 and A301-454A). We observed the same tissue expression patterns for CIP2A in the Western blot of these additional two (2) antibodies, confirming our finding and conclusion in Example 6. In sum, the studies detailed in Examples 6 and 7 show that CIP2A exhibits an increased expression in bladder cancer tissues. CIP2A protein is expressed in both low-grade TCC and high-grade TCC. The expression correlation is seen more significant in high-grade TCC.

EXAMPLE 8

### CIP2A Antibody Specificity—ShRNA Gene Silence Approach

So far, we observed that CIP2A protein is specifically expressed in bladder cancer tissue, but not in adjacent tissue from the same patient (See, Example 6). In this Example, we examined the question whether CIP2A protein is also present in urine of bladder cancer patients. This study addresses if CIP2A can be used as urine marker for early detection of bladder cancer. We determined if there CIP2A is detectable in urine; and if so, if there is a correlation of CIP2A in urine in bladder cancer patients.

In order to detect CIP2A in urine, we further obtained ten additional anti-CIP2A antibodies (other than the SC80662, SC-80660 and A301-454A) (See, Table 2). All of the antibodies are either monoclonal antibody (mAb) or polyclonal antibody (pAb), targeted against different antigen sites present on the CIP2A. All of these thirteen commercially available antibodies purportedly recognize and bind to CIP2A.

In this study, we verified the antibody specificity by first stably transfecting shRNA against CIP2A in HeLa cells (See, Experimental Methods & Procedures). After shRNA transfection, anti-CIP2A antibody binding was evaluated. The shRNA transfection in HeLa cells should abolish the anti-CIP2A antibody binding, thus illustrating antibody specificity. However, if anti-CIP2A antibody binding did not diminish after the shRNA transfection, we attributed the antibody binding as non-specific.

Out of the thirteen anti-CIP2A antibodies we examined, only seven (7) passed the specificity test (See, Table 2, labeled with “✓”). Therefore, we only employed these seven (7) specific anti-CIP2A antibodies in subsequent studies.

TABLE 2
Evaluation of Thirteen Anti-CIP2A Antibodies

| Antibodies | Cat # (Ab types) | Antigen Sites on CIP2A | ShRNA |
|---|---|---|---|
| 1 | sc-80662 (mAb) | C-terminus | ✓ |
| 2 | sc-80660 (mAb) | C-terminus | ✓ |
| 3 | A301-453A (pAb) | 550-600 AA | X |
| 4 | A301-454A (pAb) | 853-903 AA | ✓ |
| 5 | Ab61863 (mAb) | C-terminus (300 AA) | ✓ |
| 6 | C0030-03B3 (mAb) | C-terminus | ✓ |
| 7 | NB100-74663 (pAb) | Peptide between 550 and | X |
| | | 600 AA | |
| 8 | sc-80661(mAb) | C-terminus | ✓ |
| 9 | sc-80659 (mAb) | C-terminus | X |
| 11 | NB100-68264 (pAb) | 853-903 AA | ✓ |
| 12 | NB100-68263 (pAb) | Peptide between 550 and | X |
| | | 600 AA | |
| 13 | Ab84547 (pAb) | 550-600 AA | X |

### EXAMPLE 9

### Increased CIP2A Expression in Bladder Cancer

In these series of study, we determined if an increased CIP2A mRNA expression in bladder cancer is parallel to that of CIP2A protein expression. To that end, we used multiple bladder cancer cell lines (namely, RT-4, T-24, 5637 and TCCSUP;

commercially available from ATCC) as well as bladder tissues obtained from patients suffering from bladder cancer. Total cellular mRNA from cells or bladder tissues were isolated using protocols described in “Experimental Methods and Protocols.” Frozen bladder tissue samples were treated with RNAlater-ICE (Invitrogen, Calsbad, Calif.) and prepared using the RNeasy Mini Kit from Qiagen (Valencia, Calif.) according to the manufacturer's protocol. The quality and quantity of RNA was determined by spectrophotometer analysis. The purified RNA was stored in aliquots at −80° C.

CIP2A mRNA expression was quantified using qRT-PCR in the isolated mRNA obtained from either the cell lines or the bladder tissues. Reverse transcription of the isolated mRNA was performed. Specifically, Superscript III (Invitrogen, Carlsbad, Calif.) was used for the cDNA synthesis. A qRT-PCR assay was used for quantifying the CIP2A mRNA expression level using the Stratagene MX3000P system (La Jolla, Calif.) with validated primer sets purchased from Applied Biosystems (Carlsbad, Calif.) (Pre-developed TaqMan assay ID CIP2A: Hs00405413_m1). GAPDH was used as a housekeeping gene (primer sets purchased from Applied Biosystems; GAPDH: 4326317E). GAPDH was chosen based upon the use and comparison among multiple housekeeping genes such as GAPDH, beta-actin, beta-microglobulin and 18S RNA, GAPDH expression was found to be the best housekeeping gene (i.e., remained unchanged consistently throughout different experiments) when used in our qRT-PCR assay with the bladder cancer cell lines and tissues.

We performed PCR using 1× Master mix according to manufacturer's instructions which included thermocycling profile of [95° C.×3 min+(95 ° C.×30 sec+60° C.×1 min)×40]. Relative mRNA expression was determined using the ΔΔCt method normalized to GAPDH. All PCR assays were performed in triplicate. Results are the representative average of two independent reactions.

We first examined CIP2A mRNA expression in cultured cells. Notably, we could not detect any mRNA expression in normal colon fibroblast cell line (i.e., CCD-112CoN) as well as immortalized normal cervical cell line (i.e., Ect1). In contrast, when CIP2A mRNA expression was determined in the four (4) bladder cancer cell lines (i.e., RT-4, T-24, 5637 and TCCSUP), we noted that the CIP2A mRNA expression is increased in these bladder cancer cell lines (˜2-5 folds) relative to that in normal cell lines (i.e., non-cancer cells).

We next examined CIP2A mRNA expression in bladder tissues obtained from patients. Bladder tissues from 20 normal subjects were found to have minimal levels of CIP2A mRNA expression in our qRT-PCR assay. In contrast, bladder tissues from 20 bladder cancer patients exhibited an increased expression level of CIP2A mRNA. Specifically, the relative expression level of CIP2A mRNA in bladder cancer specimens is ˜2-5 folds higher than that in normal subjects (patients without bladder cancer).

Taken together, these data indicate an increased CIP2A mRNA expression in bladder tissues obtained from patients who suffered from bladder cancer.

### EXAMPLE 10

### Urine Spiking for Evaluating Anti-CIP2A Antibodies

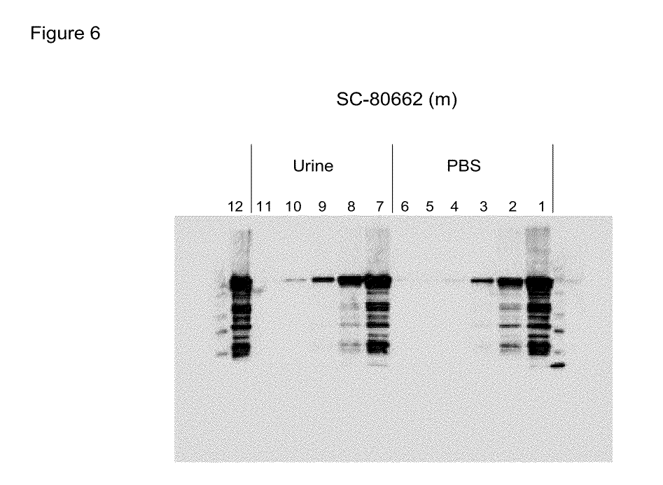

To find out if any specific anti-CIP2A antibodies would be suitable for detecting CIP2A antigen in urine, we performed a urine spiking experiment. Varying amounts of recombinant full-length CIP2A protein (i.e., flag-CIP2A) from 0.8 ng to 500 ng were spiked into 20 μl urine (control urine that did not contain CIP2A). The CIP2A spiked urine was analyzed by SDS PAGE (7.5%) in a Western blot followed by detection with the specific anti-CIP2A antibodies. The results are summarized in Table 3 and FIG. 6.

Legend for FIG. 6

| | Lane # | |
|---|---|---|
| | | magic marker |
| Pre-treated in PBS | 1 | Flag-CIP2A Elution #2, 500 ng |
| for 15 min | 2 | Flag-CIP2A Elution #2, 100 ng |
| | 3 | Flag-CIP2A Elution #2, 20 ng |
| | 4 | Flag-CIP2A Elution #2, 4 ng |
| | 5 | Flag-CIP2A Elution #2, 0.8 ng |
| | 6 | Color ladder |
| Pre-treated in urine | 7 | Flag-CIP2A Elution #2, 500 ng |
| for 15 min | 8 | Flag-CIP2A Elution #2, 100 ng |
| | 9 | Flag-CIP2A Elution #2, 20 ng |
| | 10 | Flag-CIP2A Elution #2, 4 ng |
| | 11 | Flag-CIP2A Elution #2, 0.8 ng |
| no-treatment | 12 | Flag-CIP2A Elution #2, 50 ng |
| control | | |
| | | magic marker |

Interestingly, out of the seven (7) specific CIP2A antibodies, we found that only four (4) specific CIP2A antibodies (i.e., SC-80662, SC-80660, Ab61863, and C0030-03B3) would be ideal in Western blot detection of CIP2A in urine.

TABLE 3
Urine Spiking To Evaluate Specific Anti-CIP2A Antibodies

| Cat # (types of | Antigen sites on | Antibody Detection in |
|---|---|---|
| antibodies) | CIP2A | CIP2A Spiked Urine |
| sc-80662 (mAb) | C-terminus | ✓ |
| sc-80660 (mAb) | C-terminus | ✓ |
| A301-454A (pAb) | 853-903 AA | X |
| Ab61863 (mAb) | C-terminus (300 AA) | ✓ |
| C0030-03B3 (mAb) | C-terminus | ✓ |
| sc-80661(mAb) | C-terminus | X |
| NB100-68264 (pAb) | 853-903 AA | X |

### EXAMPLE 11

### Urine Precipitating, Concentrating, and Western Blot Optimization for Evaluating Anti-CIP2A Antibodies

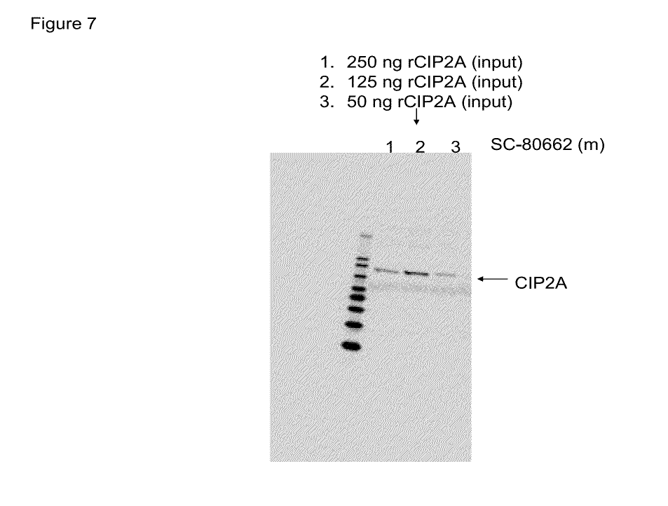

50 ng of recombinant human CIP2A was added to neat urine (control urine that does not contain any CIP2A) (FIG. 7). We optimized the CIP2A detection in urine by first concentrating urine by 100 fold. This optimization is necessary in order to detect CIP2A in human urine because CIP2A is only present in human urine in minute quantities. In this study, we concentrated neat spiked urine from 50 mL to 500 μL (acetone precipitation) by spin 1,500 g for 15 min. Then we further concentrated urine from 500 μL to 100 μL by filter column (i.e. Amicon microcon). 40 μL of final concentrated urine was loaded to 4-20% SDS PAGE and Western blot detection.

Using the concentrated urine, we proceed to determine if the four (4) antibodies would detect CIP2A protein in concentrated urine. We found that all the four (4) antibodies tested are equally good for detecting CIP2A. The detection ranges from 16 ng to 200 ng CIP2A present in 50 ml neat urine.

### EXAMPLE 12

### Test Patient Urine Samples

After the optimization, we next examined using the four (4) specific CIP2A antibodies in urine obtained from bladder cancer patient. The clinical study involved consented bladder cancer patients under Israeli Ministry of Health (Protocol no. 902008-0588) and Local Ethics Committee (Protocol no. 1091) approved protocols from Wolfson Medical Center, Holon, Israel (ClinicalTrials.gov Identifier: NCT00962052).

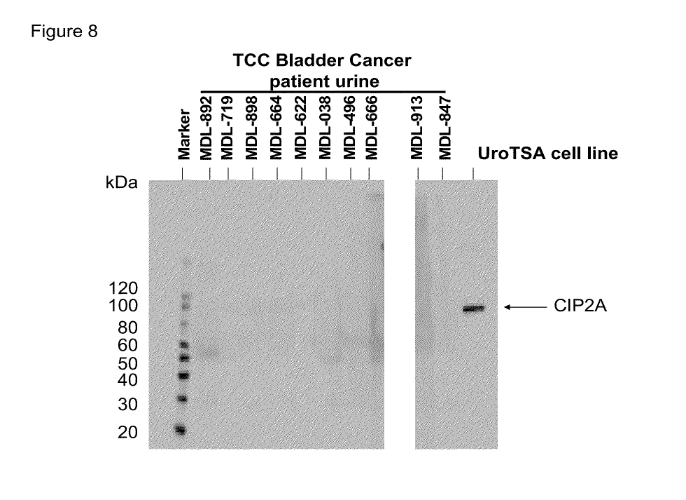

Out of the 15 urine samples obtained from bladder cancer patients, we failed to detect any CIP2A protein in their urine (See, FIG. 8 and Table 4). This data clearly indicate that while CIP2A protein expression is increased in bladder tissues of patients suffering from bladder cancer, there is no CIP2A present in urine.

TABLE 4
CIP2A expression in urine of bladder cancer patients

| | | | Tissue | Urine |
|---|---|---|---|---|
| Tissue ID | Grade | Stage | CIP2A | CIP2A |
| 4554984-7 T | High grade | TCC T1 High Grade | + | − |
| 4281689-2 T | High grade | TCC In Situ (CIS) (S/P | − | − |
| | | T1 High Grade) | | |
| 6195271-9 T | High grade | TCC T2 High Grade | + | − |
| 318044898 T | High grade | TCC T1 High Grade | − | − |
| 5371866-4 T | Low grade | TCC Ta Low Grade | − | − |
| 442262-2 | | | − | − |
| 5823003-8 T | High grade | TCC Ta High Grade | + | − |
| 6460549-6 T | High grade | TCC T1 High Grade | − | − |
| 6940766-6 T | Low grade | Previous TCC Ta Low | − | − |
| | | Malignant Potential | | |
| 465291-3 T | Low grade | TCC Ta Low | − | − |
| | | Malignant Potential | | |
| 905450-3 | Low grade | TCC Ta low grade(II) | + | − |
| 60093812-T | Low grade | T1 | − | − |
| 6541602-6 | High grade | T1 | − | − |
| 269104-6 | High grade | T2 | + | − |
| 4471371-7 | High grade | T2 | + | − |

+: CIP2A protein expression is detected
−: CIP2A protein is not expressed

### EXAMPLE 13

### Expanded Clinical Study Showing CIP2A Protein Expression in Bladder Tissues in Bladder Cancer Patients

In Example 6, we have enrolled human subjects in the clinical study so as to provide 12 normal bladder tissues and 20 cancer tissues (See, Table 1).

In this Example 12, we further expanded the clinical study to include an additional 31 normal bladder tissues and 23 cancer tissues. In this expanded clinical study, we have examined a total of 43 normal bladder tissues 43 cancer tissues.

In this expanded clinical study, we similarly examined CIP2A expression in bladder cancer tissues as compared to that in normal tissues. We obtained snap-frozen bladder tissues from normal adjacent sites (n=43) and tumor sites (n=43) from TCC bladder cancer patients (See, Table 5). Adjacent normal tissues from the same TCC subjects were obtained and used for comparison. Pathology and histo-pahological diagnose were confirmed by pathology preformed at Wolfson Medical Center, Israel. The median age of patients at diagnosis was 70 years (range 47-86). The majority of patients were male and one patient was female. Patients with low malignant potential, low grade, and high grade were 9.3%, 37.2%, and 53.5%, respectively. Patients with pTa, pT1, pT2 and pT3 or above were 37.2%, 25.6%, 20.9%, and 16.3%, respectively.

TABLE 5
Bladder Cancer Patient Characteristics

| Bladder Cancer (TCC) | Number | Percent |
|---|---|---|
| Total number | 43 | |
| Age (median) | 70 | |
| Gender | | |
| Male | 42 | 97.7 |
| Female | 1 | 2.3 |
| Grade | | |
| Low malignant potential | 4 | 9.3 |
| Low grade | 16 | 37.2 |
| High grade | 23 | 53.5 |
| Stage | | |
| pTa | 16 | 37.2 |
| pT1 | 11 | 25.6 |
| pT2 | 9 | 20.9 |
| ≧ pT3 | 7 | 16.3 |

TCC: transitional cell carcinoma

Total cell lysates were prepared from tissues using modified RIPA buffer and analyzed by Western blot assay (See, Experimental Methods & Procedures). CIP2A monoclonal antibody (SC-80662; Santa Cruz, Calif.) was used for the specific detection of CIP2A protein expression in tissues. HeLa total cell lysate extracts were used as control.

Out of the 43 bladder cancer tissues, CIP2A expression was elevated in 18 bladder cancer tissues. Of all the 43 normal adjacent tissues, none had any detectable CIP2A protein expression. We determined the sensitivity and specificity of CIP2A protein expression in bladder cancer tissue to be 42% and 100%, respectively (See, Table 6 below).

### EXAMPLE 14

### CIP2A Protein Expression is Elevated in High Risk Bladder Cancer Patients

We examined if there is a correlation between CIP2A protein expression and various bladder cancer stages and grades. The percentage of CIP2A protein positive tissue increased with the grade of bladder tumor, 0% in low malignant potential, 10% in low grade, and 65% in high grade (See, Table 6). The frequency of CIP2A expression in high grade tumor is significantly higher than that in low grade tumor (P=0.008).

The present study also indicated that CIP2A protein expression levels increased concomitantly with the progression of bladder cancer. There were significantly more CIP2A-positive cases among tumors with invasive diseases (stage pT2 and above) compared to those with non-invasive disease (stage pT1 and below) (See, Table 2, P=0.00008). Only 12% of bladder cancer patients with pTa tumor over-express CIP2A protein. These data suggest that CIP2A protein expression was less frequent in low risk and non-invasive tumors than high risk and invasive tumors allowing this protein to serve as an indication of disease progression.

TABLE 6
CIP2A Protein Expression In Bladder Cancer Tissue

| | CIP2A Protein Biomarker | | |
|---|---|---|---|
| TCC | Expression | | P |
| Overall | 42% (18/43) | | |
| Grade | | | |
| Low malignant | 0% (0/4) | | |
| potential | | | |
| Low Grade | 19% (3/16) | | 0.008 |
| High Grade | 65% (15/23) | | |
| Stage | | | |
| Non-invasive | | | |
| pTa | 12% (2/16) | 19% (5/27) | 0.00008 |
| pT1 | 27% (3/11) | | |
| Invasive | | | |
| pT2 | 67% (6/9) | 81% (13/16) | |
| ≧ pT3 | 100% (7/7) | | |

### Experimental Methods and Procedures

1. Cell Lines: Human bladder cancer cell lines (e.g., T-24 and RT-4) were maintained in McCoy's 5A medium supplemented with 10% fetal bovine serum (FBS). Human bladder cancer cell lines (e.g., 5637 and TCCSUP) were maintained in

RPMI supplemented with 10% FBS. SV-40 transformed human bladder urothelium cell line (i.e., UroTSA cell line) was maintained in DMEM medium supplemented with 10% FBS. All cell lines were maintained at 37° C. in 5% CO₂.

2. Whole Cell Lysates from Cell Lines: Prior to cell lysis, cultured cells were washed with 10 ml of cold PBS. Whole cultured cells were lysed using 1 ml of modified RIPA buffer (50 mM Tris-HCl pH 8.0, 250 mM NaCl, 1% NP-40, 0.25% sodium deoxycholate, 2 mM EDTA and protease inhibitor cocktail from Calbiochem) supplemented with protease inhibitors (Roche, Indianapolis, Ind.) at a concentration of 1 μg/μl. Lysed cells were scraped and transferred to 1.5 ml centrifuge tube and centrifuged at 14,000 rpm for 10 min. to collect supernatant (i.e., total cell lysates). Total cell lysates were collected by spinning for 10 minutes at 4° C. The protein concentration was determined by Protein Assay from Bio-Rad (Hercules, Calif.) based on the method of Bradford. Total protein lysate was stored in aliquots at −80° C.

3. Total RNA Preparation from Cultured Cell Lines: RNA was obtained using RNeasy Mini Kit from Qiagen (Valencia, Calif.). Cells were grown and collected into a pellet as described. Cells were re-suspended in 350 μl Buffer RLT and passed through a QIAshredder spin column (Qiagen; Valencia, Calif.) and centrifuged for 2 min. 350 μl 70% ethanol was added to the lysate and mixed by pipetting. The mixture was centrifuged through an RNeasy spin column for 15 seconds at 8,000×g. The column was washed once with 700 μl Buffer RW1 and twice with 500 μl Buffer RPE. After the spin column was dried by centrifuge, RNA was eluted with 50 μl RNase-free water. The quality and quantity of RNA was determined by NanoDrop analysis. RNA was stored in aliquots at −80° C.

4. Tissue Sample Collections: Frozen, cold cut tissue samples from bladder tumor and adjacent normal tissues were obtained from patients from Wolfson Medical Center (Israel) as well as from ABS Analytical Biological Services Inc. (US).

5. RNA Preparation From Bladder Tissues: RNA from cervical tissue was obtained using RNeasy Fibrous Tissue Mini Kit from Qiagen (Valencia, Calif.). Homogenized tissue powder (≦30 mg) was added to and mixed with 300 μl Buffer RLT, 590 μl RNase-free water and 10 μl proteinase K solution. The mixture was incubated at 55° C. for 10 minutes then spun down at 20-25° C. for 3 minutes at 10,000×g. The supernatant was transferred into a new micro-centrifuge tube and mixed with 450 μl 100% ethanol. The mixture was passed through an RNeasy Mini spin column at room temperature and spun for 15 seconds at 8,000×g. The column was washed with 350 μl Buffer RW1, incubated with DNase I for 15 minutes and washed again with RW1. After washing with 500 μl Buffer RPE, the RNA was eluted from column with 50 μl RNase-free water. The quality and quantity of RNA was assessed by NanoDrop analysis. The RNA was stored in aliquots at −80° C.

6. Tissue Extracts from Tissue Samples: Cold cut tissue samples were collected and immediately frozen upon removal. Samples were shipped on dried ice and stored at −80° C. To obtain tissue extract, cold cut frozen tissue samples were homogenized in RIPA buffer (400 μl) using a mortar and pestle. Homogenized tissue was centrifuged at 14,000 rpm at 4° C. for 20 min and supernatant was saved for downstream analysis. The amount of protein in each sample was quantified using a bicinchoninic acid (BCA) assay kit (Pierce, Thermo Fisher Scientific, Rockford, Ill.).

7. Analysis and Quantitation of mRNA: mRNA expression was evaluated using qRT-PCR. For each sample, one-half micro-gram (0.5 μg) of total RNA was reverse transcribed into cDNA using Superscript III (Invitrogen Life Technologies (Carlsbad, Calif.)).

A mixture containing 0.5 mg total RNA, 1 μl oligo dT primer (50 μM), 1 μl Annealing Buffer, and RNase/DNase free water to a final volume of 8 μl was prepared. This mixture was incubated at 65° C. for 5 minutes, and then immediately placed on ice for at least 1 minute. 10 μl of 2× first-strand reaction mix and 2 μl of SuperScript III/RNase OUT Enzyme Mix was added to the reaction and the reaction incubated at 50° C. for 50 minutes. The reaction was terminated by incubating at 85° C. for 5 minutes.

The qRT-PCR was performed using Stratagene (La Jolla, Calif.) Fast Real-Time PCR System with validated primer sets. All primers were purchased from Applied Biosystems (Foster City, Calif.). Thermal cycler parameters were as follows: Heated to 95° C. for 3 minutes; 40 amplification cycles at 95° C. for 30 seconds (denaturing), 60° C. for 1 minute (annealing and extension). The amount of product in a particular sample was determined by interpolation from a standard curve of cycle threshold (Ct) values generated from dilution series with known amounts of gene product. Each gene is expressed as a relative ratio of gene to the housekeeping gene GAPDH. HeLa cell was used as calibrator for the analysis. The expression level of a gene was also represented as fold increase (2^{-ΔΔCt}), where ΔΔCt=[ΔCt_{(cervical cancer)}]−[ΔCt₍ₙₒᵣₘₐₗ₎], and ΔCt=[Ct₍ₛₐₘₚₗₑ₎]−[Ct_{(GAPDH)})]. All PCR assays were performed in triplicate. Results are representative average of two reactions.

8. Western Blot: CIP2A and beta-actin monoclonal antibodies were purchased from Santa Cruz Biotechnology (Santa Cruz, Calif.) and Sigma (St. Louis, Mo.), respectively. Briefly, 50-100 μg total protein was separated by SDS-PAGE under reducing conditions and transferred to polyvinylidene fluoride (PVDF) membranes. Membranes were blocked with 5% milk in PBST (Phosphate buffered saline with 0.1% Tween-20) for one hour at room temperature. Membranes were then incubated with diluted primary antibody for 2 hours and then washed five times with PBST buffer. Membranes were incubated with peroxidase-labeled secondary antibody (goat anti-mouse IgG secondary antibody) for 90 minutes at room temperature. Goat anti-rabbit IgG diluted 1:5,000 in PBST was used for assays employing a polyclonal primary antibody. Goat anti-mouse IgG (KPL, Gaithersburg, Md.) diluted 1:5,000 in PBST was used for assays employing a monoclonal primary antibody. All membranes were visualized using enhanced chemiluminescence (ECL) detection (GE Healthcare, St. Louis, Mo.) and film exposure.

9. CIP2A shRNA Knockdown Cell Line: CIP2A knockdown cell lines were constructed using the Thermo Scientific Open Biosystems Expression Arrest GIPZ Lentiviral shRNAmir system (cat. no. RHS4430-98912354) according to the manufacturer's instructions (ThermoScientific, Huntsville, Ala.). To prepare CIP2A-ShRNA lentivirus, 5×105 293FT cells were transfected with 10 μg of pGIPZ-CIP2A shRNA and 5 μg of the packaging vectors (i.e., pCMVΔR8.2 and pHCMV-G) and grown at 37oC in 5% CO2. Supernatants of the transfected cells (containing lentivirus particles) were collected at 24 and 48 hours post-transfection. To obtain the CIP2A knockdown HeLa cell line, HeLa cells were transduced with CIP2A shRNA lentiviral particles and selected using puromycin (2.5 μg/μl ) (Sigma-Aldrich, St. Louis, Mo.).

10. Statistical Analysis: The frequency association between CIP2A protein expression and pathological status such as grade and stage of tumor, was analyzed using Fisher's Exact Probability Test. The P value was a result of a two-tailed test. A P value of <0.05 was considered as statistically significant.

What is claimed is:

1. A method of detecting CIP2A protein expression in a bladder tissue obtained from a human suspected of suffering from bladder cancer, comprising the steps of:
(a) obtaining a bladder tissue from a human;
(b) preparing a lysate from said bladder tissue; and
(c) quantifying CIP2A protein expression in said prepared lysate, wherein an increased CIP2A protein expression in said prepared lysate relative to that in a normal bladder tissue is indicative of bladder cancer in said human.

2. The method of claim 1, wherein said preparing step is performed by modified RIPA solution.

3. The method of claim 1, wherein said quantifying step for CIP2A protein expression is performed by Western blot analysis or ELISA.

4. The method of claim 1, wherein said Western blot analysis or ELISA is performed using an anti-CIP2A monoclonal antibody or polyclonal antibody.

5. The method of claim 1, wherein said bladder cancer is a high-grade transitional cell carcinoma or high-stage transitional cell carcinoma.

6. A method of detecting CIP2A mRNA expression in a bladder tissue obtained from a human suspected of suffering from bladder cancer, comprising the steps of:
(a) obtaining a bladder tissue from a human;
(b) isolating mRNA from said bladder tissue; and
(c) quantifying CIP2A mRNA expression in said bladder tissue, wherein an increased CIP2A mRNA expression in said bladder tissue relative to that from a normal bladder tissue is indicative of bladder cancer in said human.

7. The method of claim 6, wherein said mRNA isolating step is performed by guanidinium thiocyanate or phenol-chloroform.

8. The method of claim 6, wherein said quantifying step for mRNA expression is performed by qRT-PCR.

9. A kit for detecting bladder cancer in a human, comprising: a) a reagent for quantifying CIP2A expression level; and b) an instruction for use of said reagent in quantifying CIP2A expression level, wherein an increased CIP2A expression level is indicative of bladder cancer.

10. The kit of claim 9, wherein said reagent comprises an anti-CIP2A antibody to quantify the protein expression level of CIP2A.

11. The kit of claim 9, wherein said reagent comprises a forward primer and a reverse primer specific for CIP2A mRNA used in a qRT-PCR to specifically quantify the mRNA expression level of CIP2A.

12. The kit of claim 9, further comprises c) a reagent for isolating mRNA.

13. The kit of claim 11, further comprises a hybridization probe.
